(19) United States
(12) Patent Application Publication
(10) Pub. No.: US 2015/0005763 A1
(43) Pub. Date: Jan. 1, 2015

(54) GUIDE CATHETER WITH AN ANCHORING MECHANISM AND METHOD FOR INTRODUCING GUIDE CATHETER

(71) Applicant: OLYMPUS WINTER & IBE GMBH, Hamburg (DE)

(72) Inventor: German KLINK, Berlin (DE)

(21) Appl. No.: 14/312,356

(22) Filed: Jun. 23, 2014

(30) Foreign Application Priority Data
Jun. 27, 2013 (DE) 102013212448.1

(51) Int. Cl. A61B 18/14; A61M 25/06

(52) U.S. Cl. CPC A61B 18/1485; A61M 25/0662; A61B 2018/00273
USPC 606/41

(57) ABSTRACT

The invention relates to a guide catheter for inserting an applicator into body lumen along an insertion direction, including a guide sleeve, which encloses a lumen, furthermore including an anchoring mechanism, which is arranged and embodied to fasten the distal end of the guide sleeve on a wall delimiting the body lumen. Furthermore, the invention relates to a method for inserting a guide catheter into a body lumen along an insertion direction.

The invention relates to a guide catheter for inserting an applicator into body lumen along an insertion direction.

The invention furthermore relates to an electrosurgical system with a guide catheter and to a method for inserting a guide catheter into a body lumen along an insertion direction.

Guide catheters of the type set forth at the outset are known in the prior art and are used, in particular, to guide applicators therein, such as e.g. electrosurgical instruments for coagulation and/or ablation of biological tissue and/or deposits, which applicators are arranged in a mobile manner, in particular mobile in an insertion direction relative to the guide catheter, in a lumen of a guide sleeve of the guide catheter.

Here, the insertion direction is understood to be a direction in which a guide catheter is inserted into a body lumen. Due to anatomical conditions and since a guide catheter may have an embodiment that is easy to bend, the insertion direction is not necessarily an ideal straight line. Therefore, the feed direction, in which the guide catheter is substantially advanced, is considered to be the insertion direction. Therefore, the insertion direction also substantially corresponds to a longitudinal axis of the guide catheter.

Here, the guide catheter serves for inserting an applicator up to a target location or in front of a target location in the body lumen, in particular in a body tissue adjoining the body lumen. In bronchoscopy or when treating bronchial carcinoma, this target location, for example, lies in the bronchi of the lung. In order to reach the target location with a distal applicator tip, it is often necessary to puncture the body tissue adjoining the body lumen. Particularly if the target location lies in the bronchi, it is necessary to puncture a bronchial wall, which may be cartilaginous, by an applicator tip.

It is therefore an object of the present invention to specify a guide catheter and a method for inserting a guide catheter, which make it easier to bring an applicator to the target location. In particular, it is an object of the present invention to specify a guide catheter and a method for inserting a guide catheter which simplify puncturing, which is as precise as possible, of a bronchial wall.

According to the invention, this object is achieved by a guide catheter for inserting an applicator into body lumen along an insertion direction, comprising a guide sleeve, which encloses a lumen, furthermore comprising an anchoring mechanism, which is arranged and embodied to fasten the distal end of the guide sleeve on a wall delimiting the body lumen.

The invention is based on the discovery that, for bringing an applicator with pinpoint accuracy to the target location and for pinpoint puncturing of body tissue on, or in front of, the target location, it is necessary not only to align the distal end of the guide catheter with respect to the target location, but also to prevent a displacement or change in location of the previously aligned distal end of the guide catheter as a result of the puncturing forces and/or impulses which occur during puncturing.

The invention is furthermore based on the discovery that anchoring mechanisms of gripping instruments, as are known from e.g. DE 101 59 470 A1, are not suitable for anchoring the distal end of a guide catheter of the type set forth at the outset, since the guide catheter of the type set forth at the outset serves for inserting an applicator into body lumen along an insertion direction, and so a lumen in the interior of the guide sleeve of the guide catheter is to be kept clear for the insertion of the applicator and may not be occupied by a main body of a gripping device.

The forces which are to be absorbed by an anchoring when puncturing are directed against the puncturing direction and are therefore generally aligned obliquely or orthogonally with respect to the insertion direction.

The distal end of the guide catheter is preferably an open end, and so a distal end of the applicator or an applicator tip, which is also referred to as a probe tip, can emerge through the distal, open end of the guide catheter. The anchoring mechanism is preferably arranged and embodied on the guide catheter in such a way that the lumen enclosed by the guide sleeve also still has a clear cross section enabling an applicator to be guided in the region of the anchoring mechanism.

Therefore, the anchoring mechanism is preferably embodied in such a way that it does not impede or adversely influence a movement of an applicator in the guide catheter and out of the guide catheter through the distal end of the latter.

Therefore, by aligning the distal end of the guide sleeve, it is also possible to align the applicator tip, which emerges out of the distal end of the guide sleeve, in a targeted manner with respect to the target location prior to the puncturing.

In order also to be able to ensure a pinpoint alignment during and after the puncturing, the guide catheter according to the invention provides for an anchoring mechanism which, when activated, allows the distal end of the guide sleeve to be fastened or fixed, preferably in a releasable manner, to a wall delimiting the body lumen. If the anchoring mechanism is activated, the distal end of the guide sleeve is fastened to the wall; if the anchoring mechanism is deactivated, the distal end of the guide sleeve is preferably released from the wall again.

Such an anchoring of the distal end of the guide sleeve on a wall delimiting the body lumen is advantageous in that the distal end of the guide sleeve can be secured in this position—after it was inserted into the vicinity of the target location. In this manner, an unwanted and/or uncontrolled change in position of the guide catheter when puncturing the wall is avoided or reduced. Particularly in the case of puncturing a cartilaginous bronchial wall, relatively high puncturing forces and corresponding impulses occur, which may lead to a movement and/or a change in position of the guide catheter and/or of the applicator. An effect of this may be that the guide catheter originally aligned with respect to the target location is situated at a position deviating from this target position. This may have a negative effect on the treatment success. Anchoring the distal end of the guide catheter in, or on, a wall, in particular a bronchial wall, simplifies the puncturing of this wall at the precise target position.

Furthermore, the guide catheter preferably comprises a deflection mechanism which is arranged and configured to deflect a distal end of the guide sleeve with respect to the insertion direction. By means of this deflection mechanism, it is possible to align the distal end of the guide sleeve with respect to the target location, in particular with respect to a wall delimiting the body lumen, in order to bring the distal end of the guide sleeve up to, or in front of, a target location in a targeted manner.

The deflection mechanism can preferably be activated and deactivated from a proximal end of the guide catheter such that, first of all, the guide catheter can be inserted far enough along the insertion direction into the body lumen and the deflection of the distal end of the guide sleeve by activating the deflection mechanism takes place when the distal end is situated in the vicinity of the target location. An alignment of the distal end of the guide sleeve by the deflection mechanism in such a way that the distal end is aligned with respect to a wall delimiting the body lumen, in particular a bronchial wall, and preferably also contacts this wall is particularly preferred.

The deflection mechanism is preferably formed by a pulling element, such as e.g. a pull wire or a pull cable, which is connected to a distal section of the guide sleeve at at least one connection point, preferably at a plurality of connection points, and can be activated, in particular impinged upon with tension, from a proximal end of the guide catheter.

The applicator, which is also referred to as applicator probe or application probe, guided in a guide catheter can preferably have one or two electrodes on the distal section thereof, to which electrodes a radiofrequency AC voltage can be applied. Monopolar applicators only require one electrode. During the application, this one electrode interacts with a large-area return or neutral electrode, which is likewise connected to the body of a patient. For a bipolar application, provision is made for applicators with at least two electrodes. Such a bipolar electrosurgical instrument preferably has an elongate applicator shaft and two coagulation or ablation electrodes, which are arranged in succession in the longitudinal direction of the applicator shaft on the applicator shaft, respectively form a surface portion of the applicator shaft and are electrically insulated from one another by an insulator. A radiofrequency (RF) voltage with different potentials (bipolar) can be applied to such coagulation or ablation instruments, as a result of which the tissue surrounding the electrodes is heated to the extent that the body's own proteins denature. Coagulation or ablation instruments can also have a mechanically cutting/puncturing tip, for example a trocar, or a cutting electrode.

A first preferred configuration of the guide catheter provides for the anchoring mechanism to comprise at least one anchoring element, preferably two or more anchoring elements. The at least one anchoring element or the two or more anchoring elements are furthermore preferably embodied as a hook.

Furthermore, the anchoring mechanism is preferably arranged on the distal end of the guide sleeve. The anchoring element or the anchoring elements is or are preferably also arranged on, or in the vicinity of, the distal end of the guide sleeve. Since the anchoring mechanism is intended to fasten the distal end of the guide sleeve on a wall delimiting the body lumen, the arrangement of the anchoring mechanism or the anchoring elements thereof directly on, or in the vicinity of, the distal end of the guide sleeve is preferred.

The anchoring mechanism is preferably actuated by means of an activation mechanism in such a way that, by means of the activation mechanism, the anchoring mechanism can be moved from an anchoring position into a release position, and vice versa. In particular, it is preferable, in the release position, for the at least one anchoring element to be arranged proximally from the distal end of the guide sleeve. Furthermore, it is preferable, in the anchoring position, for the at least one anchoring element to be arranged distally from the distal end of the guide sleeve.

Preferably, the anchoring element or the anchoring elements is or are pushed distally beyond the distal end of the guide sleeve of the guide catheter, or protrude therebeyond, in an anchoring position, and are pulled back compared thereto in the release position such that, in the release position, the anchoring element or the anchoring elements do not project, or only project a little, beyond the distal end of the guide sleeve in the distal direction. As a result of this, it is possible to reduce the risk of tissue being damaged by the anchoring mechanism when inserting the guide catheter into a body lumen or the risk of an unwanted anchoring of the distal end occurring on the path to the target location (but still distant therefrom) in the guide sleeve. Furthermore, this allows the guide or work channel of a bronchoscope to be spared and protected from damage.

By way of example, a preferred configuration provides for the anchoring elements, in particular anchoring elements embodied as hooks, to be able to be pushed beyond the distal end of the guide sleeve for anchoring in the distal direction and for said anchoring elements to be able to be pulled back again for releasing or freeing.

A further preferred configuration provides for the anchoring mechanism and/or the at least one anchoring element to be embodied as a one-side expandable balloon. A one-side expandable balloon should be understood to mean a balloon which, in the expanded state, does not have an unchanging radius, but rather has a changeable radius, as seen from a longitudinal axis of the guide sleeve. Expressed differently, a one-side expandable balloon is preferably embodied coaxially with the guide sleeve in the non-expanded state, but preferably not aligned coaxially with respect to the guide sleeve in the expanded state. In particular, it is preferable, in the expanded state of the balloon, for the guide sleeve to be arranged closer to one inner wall region of the expanded balloon than to an inner wall region of the expanded balloon lying opposite thereto in the radial direction. The guide sleeve can also rest against an inner wall region of the expanded balloon.

A particular advantage of a one-side expandable balloon is that, as a result of the one-side expansion of the balloon and by supporting the one-side expanded balloon on a bronchial wall, the guide sleeve can be moved laterally against the opposite bronchial wall and it is therefore possible, particularly if a lateral opening is provided at the distal end of the guide sleeve, to simplify the applicator tip being brought to the target location with pinpoint accuracy.

In the release position, i.e. a contracted state of the balloon, the maximum diameter of the balloon preferably equals, or is only slightly greater than, the external diameter of the guide catheter or of the guide sleeve. In the anchoring position, i.e. in the expanded state of the balloon, the balloon preferably has an extended diameter which preferably corresponds to at most one diameter of the body lumen, in particular of a lumen in the bronchi. To this end, the balloon can preferably be expanded by means of an expansion medium. The balloon can be cooled or non-cooled. Particularly in the case of a cooled configuration of the balloon, it is preferable for the expansion medium to be guided into the balloon interior by a first lumen and for a second lumen, which is separate from the first lumen and opens into the balloon interior, to serve for returning the expansion medium to the proximal end of the balloon. Thus, it is possible to generate a circulation of the expansion medium, wherein the expansion medium preferably contains a cooling fluid, e.g. NaCl solution or the like, or is a cooling fluid. In a particularly preferred embodiment, the balloon can have an electrically conductive balloon surface which, particularly in the expanded state, can serve as a catheter electrode.

Such a one-side expandable balloon renders it possible to move the distal end of the guide sleeve to the wall delimiting a body lumen, for example a bronchial wall, which lies opposite to the direction of expansion of the balloon, and said one-side expandable balloon therefore particularly preferably has a radial or lateral opening at the distal end of the guide sleeve. Furthermore, in its anchoring position, namely the expanded position, the one-side expandable balloon prevents, or at least significantly restricts, a movement of the guide sleeve, in particular of the distal end of the guide sleeve, for example along or against the insertion direction and/or in a direction orthogonal and/or oblique thereto. Hence, by expanding the balloon, an anchoring of the distal end of the guide sleeve within the body lumen is created such that the forces, e.g. which occur when puncturing a bronchial wall, cannot lead to a movement and/or change in position of the guide catheter and/or of the applicator or lead to a significant reduction in such a movement and/or change in position.

The anchoring mechanism, preferably the at least one anchoring element, and/or an opening of the distal end of the guide sleeve can be aligned axially.

However, provision can also be made for the anchoring mechanism, preferably the at least one anchoring element, and/or an opening of the distal end of the guide sleeve to be aligned laterally and radially, respectively. In particular, such a configuration is preferable in order to be able to dispense with a deflection mechanism since the distal end can be anchored laterally or radially and the applicator tip can emerge laterally or radially from the opening of the distal end. However, a combination of a lateral or radial alignment of the anchoring mechanism, preferably the at least one anchoring element, and/or the opening of the distal end of the guide sleeve with a deflection mechanism is also possible.

Further advantages emerge if the anchoring mechanism can be fixed in the anchoring position. It is particularly preferable for it to be possible to fix the anchoring mechanism in a releasable manner in the anchoring position. By way of example, it is preferable to provide a latching mechanism, which is embodied to fix the anchoring mechanism in the anchoring position, in particular to fix it in a releasable manner.

Such a preferably releasable fixation of the anchoring mechanism in the anchoring position, for example by means of a latching mechanism, is advantageous in that the anchoring is kept in the anchoring position until it is actively released from the anchoring position again. In this manner, it is possible to prevent an unwanted release of the anchoring position. Appropriate securing or fixing of the anchoring mechanism in the anchoring position is advantageous particularly during puncturing, where high puncturing forces and/or impulses occur.

A further advantageous configuration of the guide catheter provides for the activation mechanism to be arranged on a proximal end of the guide catheter. By way of example, the activation mechanism can be arranged on a proximal handle of the guide catheter, or integrated therein. By way of example, the activation mechanism can be embodied as a lever on a proximal handle of the guide catheter.

The activation mechanism is preferably connected directly or indirectly to the anchoring mechanism. Such a connection can be brought about by various mechanisms. By way of example, the activation mechanism can be connected to the anchoring mechanism by a pulling element. The pulling element can preferably be embodied as a pull wire or pull cable. Furthermore, it may be preferable for the activation mechanism to be connected to the anchoring mechanism by means of a Bowden cable. In particular, it is also preferable for the activation mechanism to be connected to the anchoring mechanism by means of an elastic element. Preferably, provision is made for a plurality of elastic elements, by means of which the activation mechanism is connected to the anchoring mechanism. The elastic element or the elastic elements can preferably be embodied as (a) spring(s). Here, in particular, a direct or indirect connection between activation mechanism and anchoring mechanism should also be understood to mean a connection which combines a plurality of the aforementioned and/or other types of connections.

The connection between the activation mechanism at the proximal end and the anchoring mechanism at the distal end of the guide catheter is preferably brought about, as described above, by means of a pulling element, a Bowden cable and/or an elastic element. One or more springs, in particular, can be used to pretension the anchoring mechanism and/or to make an activation or deactivation dependent on the overcoming of a force. Here, the combination with a pulling element, in particular a Bowden cable, in order to ensure the force transmission from the proximal end of the guide catheter to the anchoring mechanism at the distal end of the guide catheter, is particularly preferred. By means of the force transmitted thusly, anchoring elements of the anchoring mechanism are then preferably brought from a release position into the anchoring position, for example by virtue of hooks being extended out of the distal end of the guide catheter, which hooks hook into the tissue of the bronchial wall and are then preferably pulled back again by means of a spring to the extent that there is secure fastening in the bronchial wall. Preferably, fixing of the anchoring mechanism in this position is then provided, e.g. by means of a latch, so as to secure the anchoring during the puncturing. Once the puncturing is complete, the anchoring can be released directly or it can still be kept there during the treatment of the body tissue at the target location by means of the applicator and only be released again when the applicator is removed.

In accordance with a further aspect of the invention, the object set forth at the outset is achieved by an electrosurgical system with an above-described guide catheter and an applicator, which is embodied as an electrosurgical instrument and guided, in a mobile manner, in the guide sleeve of the guide catheter.

In accordance with a further aspect of the present invention, the object set forth at the outset is achieved by a method for inserting a guide catheter into a body lumen along an insertion direction, comprising the following steps: inserting a distal end of a guide sleeve into a body lumen along the insertion direction, anchoring the distal end of the guide sleeve on a wall delimiting the body lumen.

The method is particularly suitable for inserting an above-described guide catheter or an above-described electrosurgical system at, or in front of, a target location in a body tissue adjoining a body lumen.

The electrosurgical system and the developments thereof and the method and the developments thereof preferably have features or method steps which make them particularly suitable for being used with a guide catheter according to the invention and the developments thereof.

In respect of the advantages, embodiment variants and configuration details of the electrosurgical system and the developments thereof, and of the method and the developments thereof, reference is made to the above description in respect of the corresponding features of the guide catheter.

A preferred embodiment of the invention is described in an exemplary manner on the basis of the attached figures. In detail:

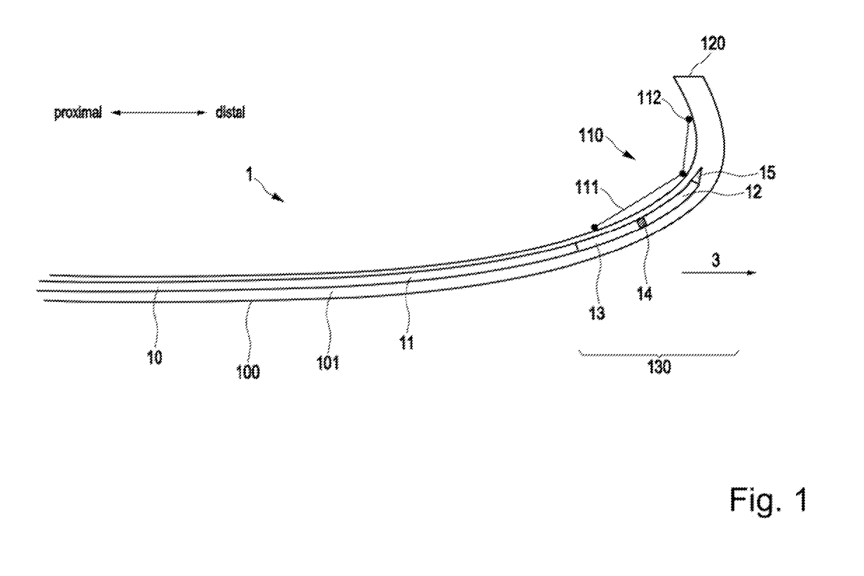

FIG. 1 shows a guide catheter with a deflection mechanism;

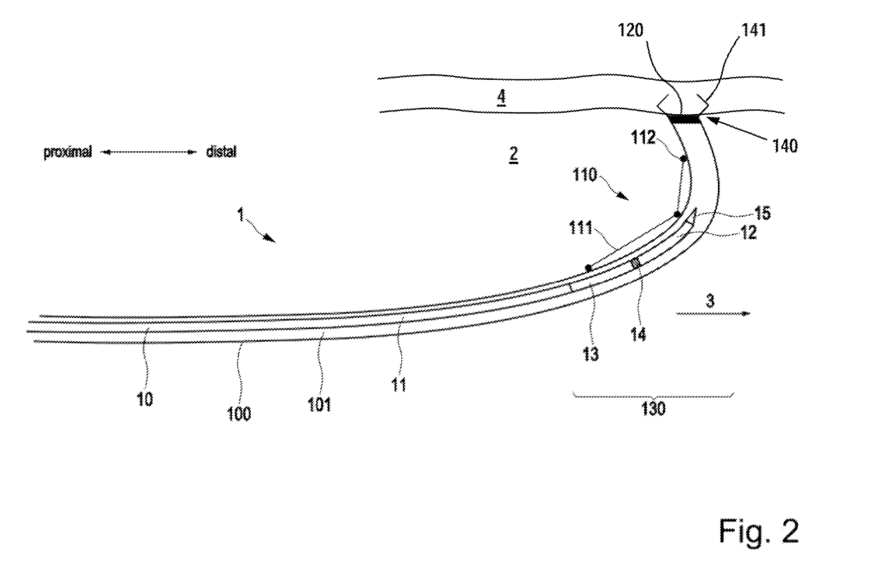

FIG. 2 shows the guide catheter with a deflection mechanism as per FIG. 1 with an activated anchoring mechanism;

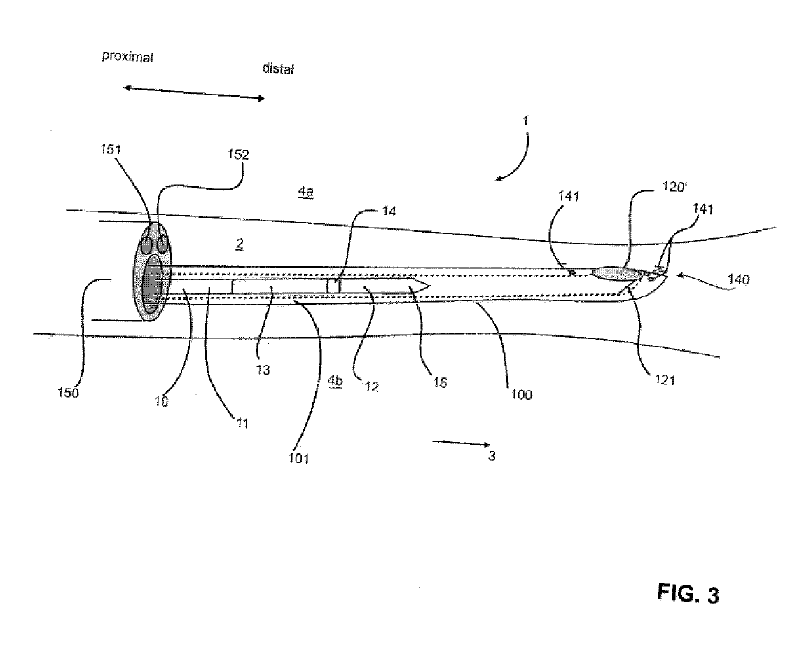

FIG. 3 shows a guide catheter without a deflection mechanism with a lateral or radial anchoring mechanism; and

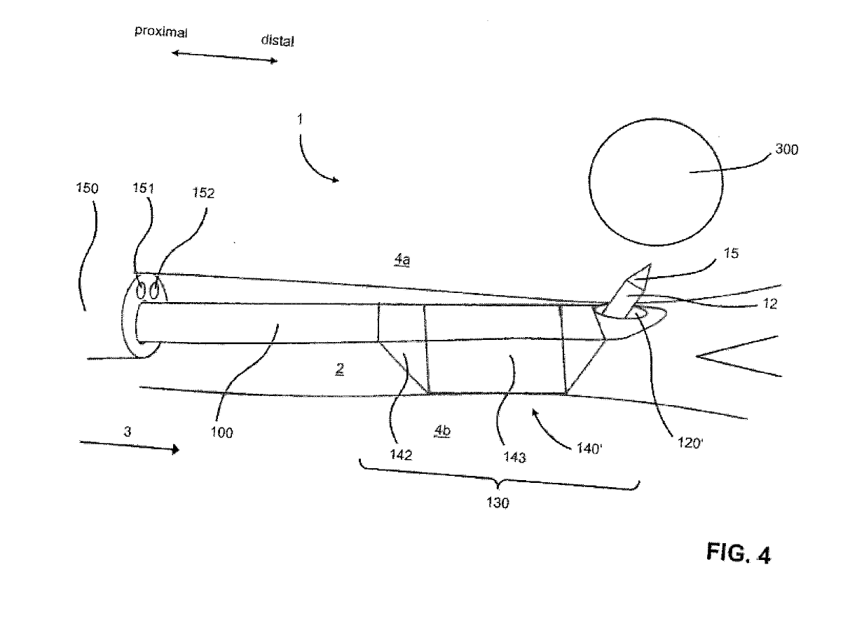

FIG. 4 shows a guide catheter with an anchoring mechanism embodied as a one-side expandable balloon.

The figures depict a guide catheter 1 for inserting an applicator 10 into a body lumen 2 along an insertion direction 3. The guide catheter 1 comprises a guide sleeve 100 which encloses a lumen 101. Elements which are equivalent or substantially functionally equivalent have been provided with the same reference signs. If nothing else is stated, the following description relates to all figures.

An applicator 10 is arranged in a movable manner, in particular in a manner movable relative to the guide catheter in the insertion direction 3, in the lumen 101 of the guide catheter 1. The applicator 10 is formed as a bipolar electrosurgical instrument with an elongate applicator shaft 11 and two coagulation or ablation electrodes 12, 13, which are arranged in succession in the longitudinal direction of the applicator shaft on the applicator shaft 11 and each form a surface portion of the applicator shaft 11. The distal electrode 12 and the proximal electrode 13 are electrically insulated from one another by an insulator 14. The insulator 14 is arranged coaxially with respect to the electrodes 12, 13 and likewise forms a surface portion of the applicator shaft 11. An applicator tip 15 is formed on the distal coagulation or ablation electrode 12. By way of example, a mechanically cutting and/or puncturing tip, for example a trocar, can be arranged on the distal applicator tip 15. A cutting electrode can also be provided for on the applicator tip 15. Overall, with the exception of the applicator tip 15, a cylindrical design with a substantially constant circular cross section emerges for the applicator shaft 11.

The distal end 120, 120′ of the guide sleeve 100 preferably has an open embodiment such that a distal end of the applicator 10 with an applicator tip 15 can emerge from the distal end 120, 120′ of the guide sleeve 100 in order to be able to advance to a target location, denoted by 300 in FIG. 4, in the tissue. In FIGS. 1 and 2, the opening of the distal end 120 is shown as an axial opening. However, the opening can also have a lateral or radial embodiment. Such a lateral or radial opening of the distal end 120′ of the guide sleeve 100 is depicted in the exemplary embodiments according to FIGS. 3 and 4.

As depicted in FIG. 3, a deflection plate 121 is arranged in the lumen 101, preferably at the distal end 120′ of the guide sleeve 100, which deflection plate serves, in particular, to deflect the applicator tip 15 such that the latter can emerge through the lateral or radial opening of the distal end 120′ without damaging the distal end 120′ of the guide sleeve 100. The deflection plate 121 can be unchanging in terms of its position, or else it can be movable, in particular deflectable or pivotable. In the case of a movable embodiment of the deflection plate, it is furthermore preferable for it also to be possible to fix the deflection plate in a deflected or pivoted position.

The guide catheter 1 can furthermore comprise a deflection mechanism 110, by means of which the distal end 120 of the guide sleeve can be deflected with respect to the insertion direction 3, as depicted in FIGS. 1 and 2. To this end, in the exemplary embodiment depicted in FIGS. 1 and 2, three connection points 112 are provided on the distal section 130 of the guide sleeve 100, at which connection points a pulling element, embodied as a pull wire 111, is connected to the distal section 130 of the guide sleeve 100. Tension can preferably be applied to the deflection mechanism 110 from a proximal end of the guide catheter. To this end, the pull wire 111 can, for example, be guided as far as the proximal end of the guide catheter (within or outside of the guide sleeve 100). By applying tension to the pull wire 111, the distal end 120 is deflected, as depicted in FIGS. 1 and 2, and, as can be identified in FIG. 2, it can be aligned in the direction of a wall 4 and preferably be brought into contact with this wall 4.

As a result of the lateral or radial opening 120′ of the exemplary embodiments shown in FIGS. 3 and 4, it is possible to dispense with a separate deflection mechanism. However, provision can also be made for a combination of deflection mechanism lateral or radial opening of the distal end of the guide sleeve. In FIGS. 3 and 4, a rinsing channel 151 and an optical channel 152 are furthermore depicted; these are preferably combined together with the guide sleeve 100 of the applicator 10 in an electrosurgical instrument, for example in a bronchoscope 150. In addition to the bronchial wall 4a, behind which the target location (a tumor 300 in FIG. 4) is situated in the examples shown in FIGS. 3 and 4, it is also possible to identify an opposite bronchial wall 4b.

The applicator 10, in particular the applicator shaft 11, preferably has an embodiment that is easy to bend in order to be able to follow a deflection of a distal section 130 of the guide sleeve 100 by means of the deflection mechanism 110.

The target location, at which a treatment is intended to be performed by means of the applicator 10, is preferably situated in the region behind the contact point of the distal end 120, 120′ of the guide sleeve 100 on the bronchial wall 4, 4a, as denoted by 300 in FIG. 4.

The wall, preferably a bronchial wall, is punctured by means of the applicator tip 15 in order to let the applicator 10 penetrate into the wall 4, 4a and hence let it advance to the target location. High puncturing forces and impulses which may lead to a displacement or change in position of the distal end 120, 120′ of the guide catheter 1 can occur, particularly when puncturing a bronchial wall.

An anchoring mechanism 140, 140′ is provided to prevent this. The anchoring mechanism 140 depicted in FIGS. 2 and 3 serves to fasten the distal end 120, 120′ of the guide sleeve 100 in the wall 4, 4a. To this end, two hooks 141 are provided in the exemplary embodiment depicted in FIG. 2, which hooks can be brought into the anchoring position depicted in FIG. 2 by means of an activation mechanism (not depicted here). In the exemplary embodiment depicted in FIG. 3, three hooks 141 are provided, which can likewise be brought into the anchoring position depicted in FIG. 3 by means of an activation mechanism (not depicted here). In each case, provision can also be made for more or fewer hooks. The anchoring elements, embodied as hooks 141, of the anchoring mechanism 140 can be pushed out of the distal end 120, 120′ of the guide sleeve 100, preferably by means of an activation mechanism embodied as a Bowden cable, and can preferably be pulled back again in a resilient manner for the purposes of anchoring in the inner bronchial wall. In order to release the connection, the hooks 141 can be reset again, preferably by an actuation at the proximal end of the guide catheter 1, for example by actuating a lever on a handle (not depicted here), and can thus detach from the superficial anchoring thereof in the bronchial wall 4, 4a. The guide catheter 1 can then be removed from the body lumen 2 again.

The anchoring mechanism 140 depicted in FIG. 4 fixes the distal end 120′ of the guide sleeve 100 on the bronchial wall 4a. As a result of the one-side expansion of the balloon 142, the distal end 120′ is brought into contact with the bronchial wall 4a in the vicinity of the target location 300. It is possible to ensure that an unwanted movement and/or change in position of the distal end 120′ when puncturing the bronchial wall 4a is prevented or reduced to a sufficient extent by, firstly, the large-area application of the expanded balloon 142 in the anchoring position and, secondly, by the contact of the distal end 120′ of the guide sleeve 100 on the wall 4a.

As a result of such fixing of the distal end 120, 120′ of the guide sleeve 100 of the guide catheter 1 on the bronchial wall 4, 4a by means of the anchoring mechanism 140, 140′, there can also be large puncturing forces and impulses, without this leading to an unwanted and/or uncontrolled change in position of the distal end 120, 120′ of the guide catheter 1. In this manner, the applicator 10 to be guided in the guide catheter, in particular the applicator tip 15, can be brought to the target location in the body tissue in a targeted manner with increased reliability.

### LIST OF REFERENCE SIGNS

1 Guide catheter

2 Body lumen

3 Insertion direction

4, 4a, 4b Wall delimiting the body lumen

10 Applicator

11 Applicator shaft

12 Distal coagulation or ablation electrode

13 Proximal coagulation or ablation electrode

14 Insulator

15 Applicator tip

100 Guide sleeve

101 Lumen of the guide sleeve

110 Deflection mechanism

111 Pulling element embodied as a pull wire

112 Connection points

120, 120′ Distal end of the guide sleeve

121 Deflection plate

130 Distal section of the guide sleeve

140, 140′ Anchoring mechanism

141 Anchoring elements embodied as hooks

142 One-side expandable balloon

143 Electrically conductive balloon surface

150 Bronchoscope

151 Rinsing channel

152 Optical channel

300 Target location/tumor

1. A guide catheter for inserting an applicator into body lumen along an insertion direction, comprising
a guide sleeve, which encloses a lumen,
furthermore comprising an anchoring mechanism, which is arranged and embodied to fasten the distal end of the guide sleeve on a wall delimiting the body lumen.

2. The guide catheter according to claim 1,
wherein the anchoring mechanism comprises at least one anchoring element.

3. The guide catheter according to claim 2,
wherein at least one anchoring element is arranged on the distal end of the guide sleeve.

4. The guide catheter according to claim 1,
comprising an activation mechanism, which is embodied to move the anchoring mechanism from an anchoring position into a release position, and vice versa.

5. The guide catheter according to claim 2,
wherein the release position, the at least one anchoring element is arranged proximally from the distal end of the guide sleeve.

6. The guide catheter according to claim 2,
wherein the anchoring position, the at least one anchoring element is arranged distally from the distal end of the guide sleeve.

7. The guide catheter according to claim 1,
wherein the anchoring mechanism and/or the at least one anchoring element is embodied as a one-side expandable balloon.

8. The guide catheter according to claim 1,
wherein the anchoring mechanism and/or an opening of the distal end of the guide sleeve is or are aligned laterally and radially, respectively.

9. The guide catheter according to claim 1,
wherein the anchoring mechanism can be fixed in the anchoring position.

10. The guide catheter according to claim 1,
comprising a latching mechanism, which is embodied to fix the anchoring mechanism in the anchoring position.

11. The guide catheter according to claim 4,
wherein the activation mechanism is arranged on a proximal end of the guide catheter.

12. The guide catheter according to claim 4,
wherein the activation mechanism is connected to the anchoring mechanism by a pulling element.

13. The guide catheter according to claim 12,
wherein the pulling element is embodied as a pull wire.

14. The guide catheter according to claim 4,
wherein the activation mechanism is connected to the anchoring mechanism by means of a Bowden cable.

15. The guide catheter according to claim 4,
wherein the activation mechanism is connected to the anchoring mechanism by means of an elastic element.

16. An electrosurgical system with a guide catheter according to claim 1 and an applicator, which is embodied as an electrosurgical instrument and guided, in a mobile manner, in the guide sleeve of the guide catheter.

17. A method for inserting a guide catheter into a body lumen along an insertion direction, comprising the following steps:
inserting a distal end of a guide sleeve into a body lumen along the insertion direction,
anchoring the distal end of the guide sleeve on a wall delimiting the body lumen.
